(19) United States
(12) Patent Application Publication
(10) Pub. No.: US 2007/0062111 A1
(43) Pub. Date: Mar. 22, 2007

(54) System for forming roots on a plant cutting

(76) Inventor: Darren Phillips, Sacramento, CA (US)

Correspondence Address: THOMAS R. LAMPE;BIELEN, LAMPE & THOEMING, 1390 WILLOW PASS ROAD, SUITE 1020, CONCORD, CA, 94520, US

(21) Appl. No.: 11/204,139

(22) Filed: Aug. 15, 2005

(51) Int. Cl. A01H 3/00

(52) U.S. Cl. 47/58.1 R

(57) ABSTRACT

Roots on a plant cutting are formed by continuously circulating oxygenated water low in minerals and contaminates, a pH within a predetermined pH range, and a water temperature within a predetermined water temperature range about the severed end of the plant cutting.

### TECHNICAL FIELD

This invention relates to both a method and an apparatus utilized to form roots on a plant cutting.

### BACKGROUND OF THE INVENTION

Major differences exist between a plant cutting and a plant, these being considered two different horticultural categories, just as seeds and plants are considered different horticultural categories.

A cutting is a section or portion of a plant removed for propagation or other purposes. A cutting has the ability to grow its own roots when taken from a clonable plant if cared for in a properly controlled environment. A plant cutting requires strict environmental controls and standards to result in continued life and root formation, while a healthy rooted plant normally does not require such strict environments.

The formation of roots on cuttings so that they will become viable plants is far different than growing plants with roots. A seedling, which already has root structure, can endure harsher and more varied environments than a cutting having no roots, because it already has the means to efficiently take in nutrients and water. The roots of a plant are its life line.

Quick root formation for cuttings is highly important. A small section from a plant or branch top or tip of a clonable plant has the ability to form roots in most media that it is planted in, but only if the environmental factors are right both inside and outside of the particular medium employed. A change inside the medium will usually require an alteration in the outside environment. The closer you get to the perfect environment the better success rate one has in getting plant cuttings to form new roots quickly.

The longer it takes a cutting to form roots the less chance it has to survive. After it is cut from the parent plant it does not have the same ability to intake water and nutrients as efficiently as a plant with roots, and a fresh wound is susceptible to disease. For a new cutting to survive, it must mend and grow new roots so that it can again intake water and nutrients efficiently. To accomplish this, the cutting uses its own stored energy and if root formation is slow because environmental factors are not right, the stored energy will be depleted and a cutting will turn yellow, becoming weak and making it susceptible to disease. Eventually it will die.

Typically a cutting will not survive in a normal plant habitat where it is exposed to high or low temperatures, wind, rain, insects and intense light. For example, if one were to cut the top from a plant growing outdoors in the sun, in temperatures of 90 degrees or more, with the wind blowing at 15 mph, a cutting planted in the same dirt occupied by the parent plant and located next to the parent plant exposed to the same environment and provided with water will surely die in minutes if not seconds.

Plants send out roots in search of food and water, and while doing this the roots form a complex structure that can cover a large area and volume. A plant with roots has the ability to maintain a hold on the dirt, rock and sediment in which it grows. This root mass, as it grows, basically ties itself up in the growing medium, in the sense becoming part of the medium. This firm grasp in the medium makes the plant stable in the medium, enabling the plant to withstand harsh environments and giving the plant the ability to easily absorb and process the food and water through its roots from the area occupied by the root mass.

It is known in the prior art to immerse roots of plants in circulating aerated water to grow the rooted plants. Representative arrangements of this type are disclosed in U.S. Pat. No. 4,315,381, U.S. Pat. No. 5,056,260, U.S. Pat. No. 6,233,870, U.S. Pat. No. 4,096,663 and U.S. Pat. No. 4,245,433. Such arrangements are not suitable for forming roots on a plant cutting.

Some of the known techniques include the need for high humidity, conducive to mold formation and requiring tedious watering. Another known technique employs a reservoir with a large water pump that has a tendency to overheat the water and as a result increases the incidence of stem rot which makes the technique more sensitive to warmer external temperatures. Increased water temperature will reduce the amount of oxygen the water can hold exponentially. Other plant growing techniques employ media filled with water, providing no circulation and becoming stagnant if not changed regularly, such as daily. This approach is conducive to stem suffocation and stem rot because of a lack of oxygen around the stem.

As will be seen below, the approach of the present invention employs a pump as part of the novel overall system to circulate water in a desired fashion and further contributing to oxygenation of the circulated water about the lower end of plant cuttings. Impeller pump-type aerators per se employed in other environments, such as aquariums, are known. See, for example, U.S. Pat. No. 6,655,663, U.S. Pat. No. 5,988,600 and U.S. Pat. No. 5,582,777.

### DISCLOSURE OF INVENTION

The method and apparatus of the present invention provide an extremely high success of root formation as compared to prior art approaches, the success being a result of establishing a highly controlled specific environment developed after considerable research and testing by the inventor.

The method of forming roots on a plant cutting according to the teachings of the present invention includes the step of at least partially filling the interior of a receptacle with water low in minerals and contaminates, a pH within a determined pH range, and a water temperature within a predetermined water temperature range.

Oxygen is injected into the water in the receptacle interior to oxygenate the water.

A plant cutting is severed from a clonable plant and the severed end of the plant cutting is promptly placed into the oxygenated water. The plant cutting is supported while maintaining the severed end in the oxygenated water.

According to the method, continuous movement of the oxygenated water is caused while maintaining the oxygenated water in engagement with the severed end of the supported plant cutting and within the predetermined pH and water temperature ranges.

The apparatus of the invention includes a receptacle defining an interior at least partially filled with water low in minerals and contaminates, a pH within a predetermined pH range, and a water temperature within a predetermined water temperature range.

Oxygen injection structure is provided for injecting oxygen into the water in the receptacle interior to oxygenate the water. A plant cutting support is provided for supporting the plant cutting while maintaining the severed end thereof in the oxygenated water.

Water moving structure is incorporated in the apparatus for causing continuous movement of the oxygenated water while maintaining the oxygenated water in engagement with the severed end of the supported plant cutting and within the predetermined pH and water temperature ranges.

Other features, advantages and objects of the present invention will become apparent with reference to the following description and accompanying drawings.

### BRIEF DESCRIPTION OF DRAWINGS

FIG. 1 is a perspective view of apparatus constructed in accordance with the teachings of the present invention in assembled condition;

FIG. 2 is an exploded, perspective view of the apparatus;

FIGS. 3-5 are enlarged, perspective views of a plant cutting during sequential steps of the method of the invention;

FIG. 6 is a cross-sectional, elevational view illustrating a plurality of plant cuttings in place in a portion of the apparatus of the invention and showing the cuttings in partial rooted condition and subject to continued exposure of the cutting lower ends to oxygenated circulating water issuing from a water pump;

FIG. 7 is an elevational view of the apparatus with the lid or cover removed and showing by means of arrows whirlpool water circulation caused by the water pump of the apparatus;

FIG. 8 is a perspective view of the apparatus having a plurality of plant cuttings in place in and over the receptacle; and

FIGS. 9 and 10 are, respectively, perspective and plan views of an alternative form of cutting holder for use in the system.

### MODES FOR CARRYING OUT THE INVENTION

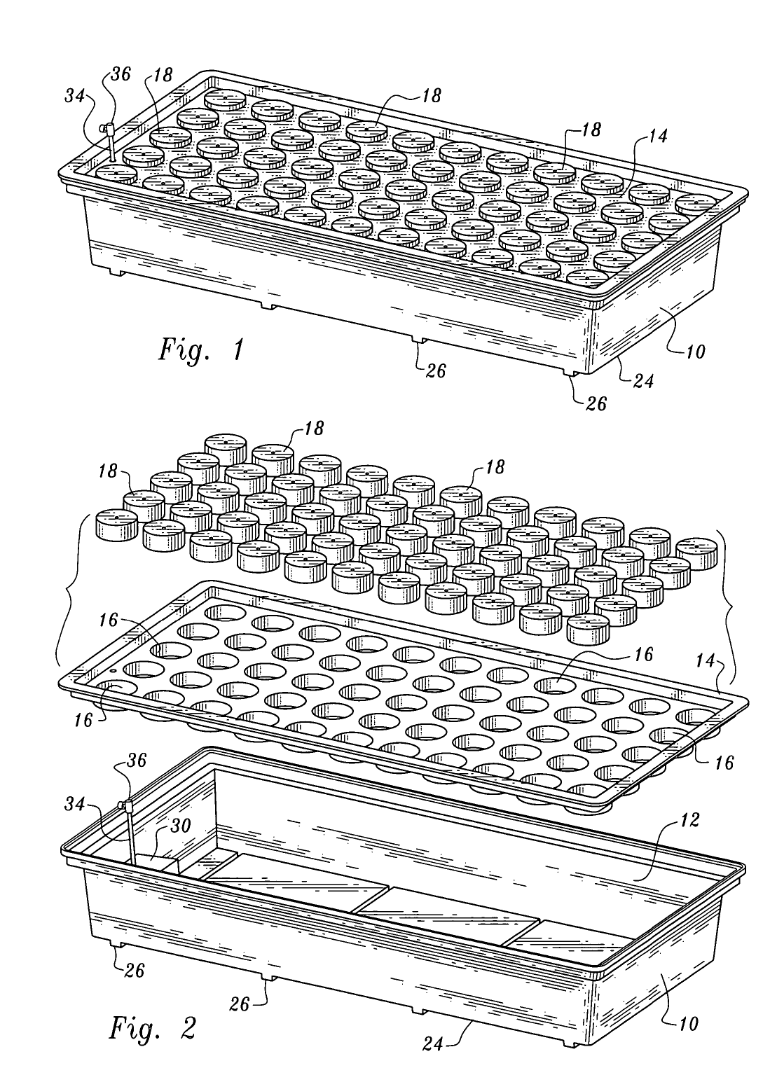

FIGS. 1-8 disclose a preferred form of apparatus constructed in accordance with the teachings of the present invention. The apparatus includes an open-topped, tray-like receptacle 10 defining an interior 12 for containing water. The receptacle 10 may be formed of molded plastic and preferably has a low profile to fit in small areas.

A removable cover 14 defining a plurality of openings 16 is positionable on the receptacle. Cutting holders 18 suitably formed of foam plastic and having central apertures 20 therein are selectively releasably positionable in openings 16. The cover and holders 18 are for the purpose of supporting plant cuttings 22. Frictional engagement between the holders and the cuttings in apertures 20 retains the cuttings at the desired locations relative to the holders, the cover and the receptacle. The cuttings may be manually slid up and down to adjust positioning, if desired. The holders 18 are compressed by the circular peripheral walls of the cover at the openings to maintain the holders in position relative to the cover. The cover has an outer flange to maintain it in position over the tray-like receptacle.

The receptacle has a bottom wall 24 and incorporates downwardly extending projections or legs 26 which are utilized to maintain more than a majority of the receptacle bottom wall out of engagement with a support surface 28 (FIG. 6) supporting the receptacle to provide thermal insulation between the receptacle and the support surface. This minimizes the absorption of surface temperatures and allows air to pass underneath the bottom wall 24, maintaining temperatures within the receptacle close to ambient outside temperatures. If one were, for example, to place the reservoir in an indoor closet on a shelf, and the shelf was attached to a wall exposed at the opposite side thereof to sunlight all day, the wall would become hot and the shelf would absorb the wall's heat, in turn, transferring the heat to whatever is supported by the shelf.

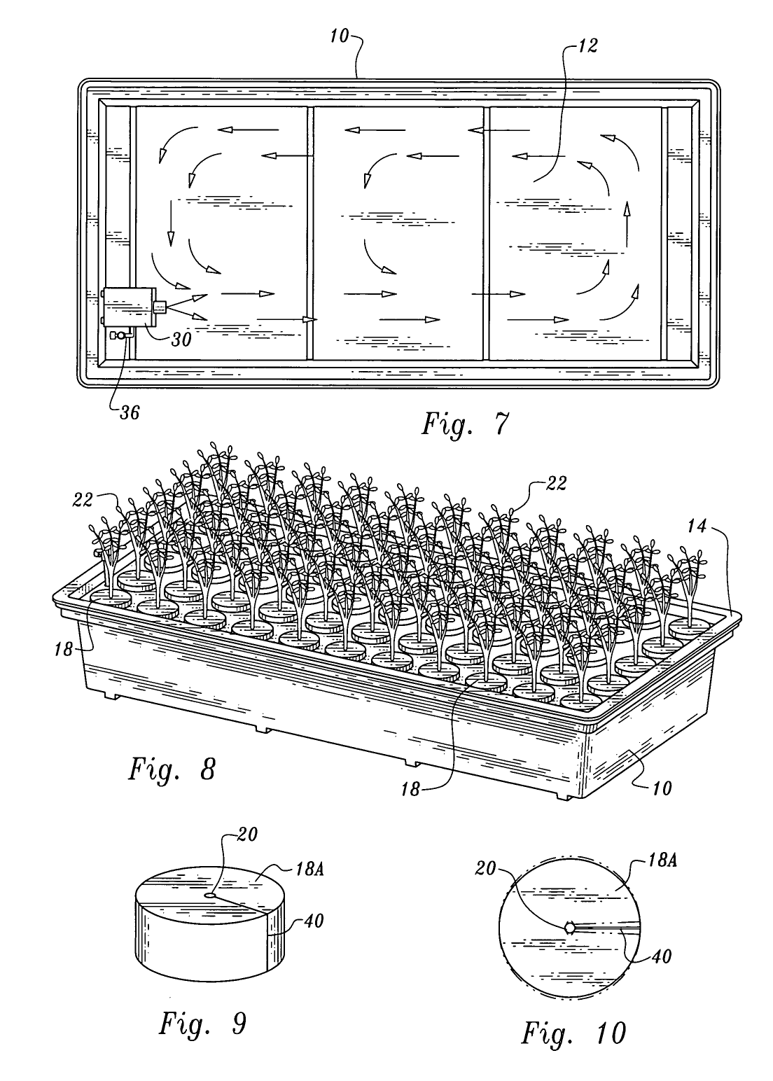

Located within the interior of the tray-like receptacle 10 is a water pump 30 having an inwardly directed nozzle 32 positioned closely adjacent to one of the side walls of the receptacle. The water pump 30 when actuated circulates water in the receptacle interior, as shown in FIGS. 6 and 7, by creating a jet of water within the interior exerting water shear forces causing a whirlpool motion as shown by the arrows in FIGS. 6 and 7.

In addition, the water pump 30 serves to oxygenate the water by injecting oxygen into the water in the receptacle. The water pump 30 receives ambient air through an air inlet conduit 34 having an air flow regulator 36 operatively associated therewith. The water pump exits the nozzle 32 as tiny bubbles entrained in the jet of water. This is shown in FIG. 6.

In accordance with the method of the invention, the interior of the receptacle 10 is filled substantially up to the level of the cover with water low in minerals and contaminates, a pH within a predetermined pH range, and a water temperature within a predetermined water temperature range.

In particular, the predetermined pH range is between about 5.5 and 7.0. The predetermined water temperature range is between about 60 degrees Fahrenheit and 90 degrees Fahrenheit. The minerals and contaminates of the water are in the range of 0 parts per million to about 300 parts per million.

In addition, ambient air about the receptacle is maintained within a temperature range of from about 70 degrees Fahrenheit to about 85 degrees Fahrenheit.

Operation of the water pump 30 injects oxygen into the water in the receptacle interior to oxygenate the water. This is a result of the water pump drawing in a controlled amount of fresh air by means of the intake or vacuum side of the pump, the injected air being within the desired outside ambient temperature range indicated above. The air bubbles produced by the water pump are emitted and circulated as described above to provide a sufficient amount of oxygen to cuttings before and after the formation of roots. The bubbles, being maintained at the desired outside temperature range approximating the water temperature, will also tend to maintain the water within the desired temperature range. The apparatus continually supplies fresh oxygen to the water and at the same time keeps the water circulating. The water pump can be placed into the water, as shown, or pump water outside of the receptacle. Experiments with the water pump outlet in different locations show differences in root growth within the interior of the receptacle. There is better uniformity of root growth and quicker results when the pump outlet is placed so that it causes the disclosed whirlpool technique.

In practice, one or more plant cuttings 22 are severed from a clonable plant. The preparation of cuttings and positioning thereof in the apparatus must be done promptly because of the cuttings' demand for water, or they will wilt and not recover. Any time a cutting starts to go limp, the cut end will need to be placed in water for a few seconds to revive it.

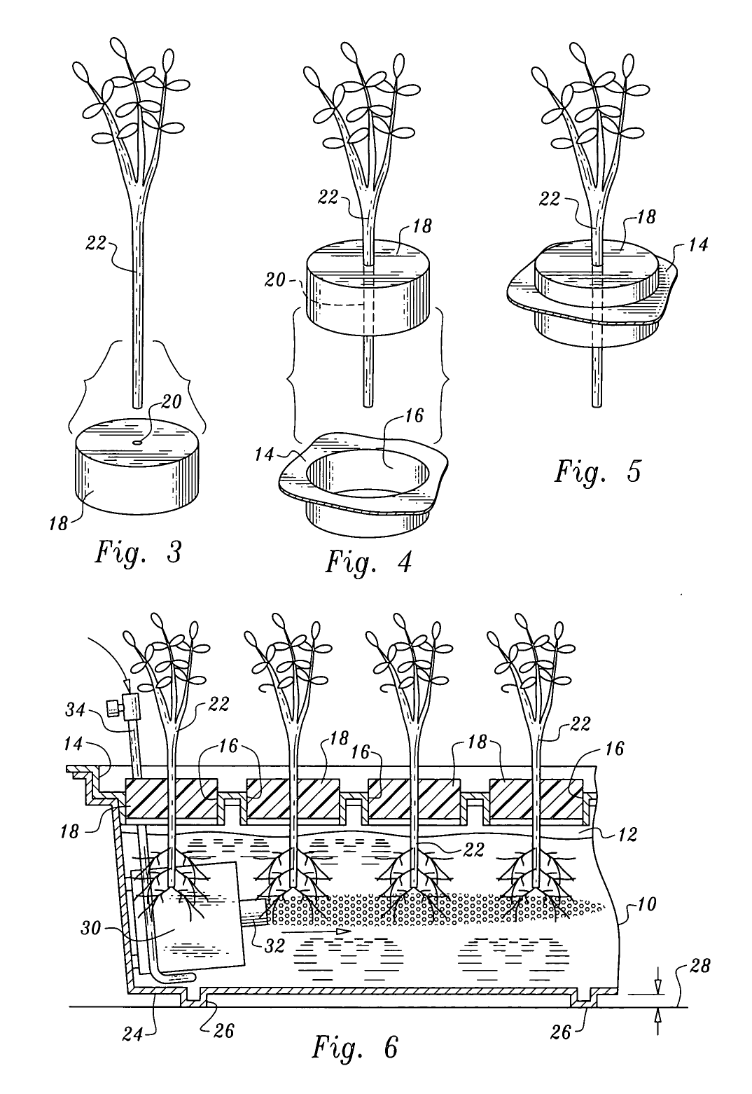

As an example, using a clonable plant that is healthy and insect free, a 4 to 6 inch cutting is severed from the top or tip of a branch or plant. The cutting is dipped into the preadjusted water in the receptacle interior quickly. This is accomplished by trimming the end branching as required to produce a clean stem as shown in FIG. 3. The stem is then passed through an aperture 20 of a holder 18, as shown in FIG. 4, and the holder and cutting are placed into position on the cover or lid 14 with the lower ends of the cutting immersed in the circulating water. This is repeated for each cutting. The lower portions of the cloned cuttings should remain submerged in the oxygen rich and circulating water through the entire rooting process. The apparatus is then preferably placed under low-level lighting until the cuttings develop roots.

After the cuttings have formed roots, as shown for example in FIG. 6, they can be planted in a medium of choice and grown to maturity. This procedure will produce a genetic copy of the plant the cutting was taken from. The water reservoir preferably runs slightly warmer than the outside air temperature so it will maintain ideal water temperatures, allowing the water to hold more oxygen and reduce any chances of stem rot and to provide quick root formation. If desired, a thermostat controlled heater (not shown) may be positioned in the receptacle. Utilizing this invention during testing with various species of plants, small roots have developed in as little as 3-5 days. The length of time of course depends upon the type of plant and strain used. The predetermined pH and water temperature ranges are maintained at all stages of the operation.

FIGS. 9 and 10 show an alternative form of holder, holder 18A, which is the same as holder 18 but includes a slit 40 communicating with aperture 20 for facilitating placement of the cutting in aperture 20 and removal therefrom. Dash lines in FIG. 10 depict how the slit may be widened by exerting opposed pulling forces on the holder.

The invention claimed is:

1. A method of forming roots on a plant cutting, said method comprising the steps of:
at least partially filling the interior of a receptacle with water low in minerals and contaminates, a pH within a predetermined pH range, and a water temperature within a predetermined water temperature range;
injecting oxygen into the water in the receptacle interior to oxygenate the water;
severing a plant cutting from a clonable plant;
promptly placing the severed end of the plant cutting into the oxygenated water;
supporting the plant cutting while maintaining the severed end in the oxygenated water; and
causing continuous movement of the oxygenated water while maintaining the oxygenated water in engagement with the severed end of the supported plant cutting and within said predetermined pH and water temperature ranges.

2. The method according to claim 1 wherein said predetermined pH range is between about 5.5 and 7.0.

3. The method according to claim 1 wherein said predetermined water temperature range is between about 60 degrees Fahrenheit and 90 degrees Fahrenheit.

4. The method according to claim 1 wherein the minerals and contaminates of said water are in the range of 0 parts per million to about 300 parts per million.

5. The method according to claim 1 additionally comprising the step of maintaining ambient air about a portion of the plant cutting disposed above the oxygenated water within a temperature range of from about 70 degrees Fahrenheit to about 85 degrees Fahrenheit.

6. The method according to claim 1 wherein said receptacle has a receptacle bottom wall and incorporates downwardly extending projections, said method including the step of utilizing said downwardly extending projections to maintain more than a majority of said receptacle bottom wall out of engagement with a support surface supporting said receptacle to provide thermal insulation between said receptacle and said support surface.

7. The method according to claim 1 wherein said water moves in a whirlpool motion within said receptacle.

8. The method according to claim 7 wherein a water pump is employed to create a jet of water within the interior of the receptacle exerting water shear forces causing said whirlpool motion.

9. The method according to claim 1 wherein a cover defining an opening is disposed over said receptacle and wherein said step of supporting said plant cutting is carried out by retaining said plant cutting in a holder releasably connected to said cover at said opening.

10. The method according to claim 8 wherein said oxygen is injected into the water in the receptacle interior by said water pump in the form of air bubbles in said jet of water.

11. The method according to claim 8 wherein said jet of water is at least partially comprised of water recirculated between said water pump and the interior of said receptacle.

12. Apparatus for forming roots on a plant cutting, said apparatus comprising, in combination:
a receptacle defining an interior at least partially filled with water low in minerals and contaminates, a pH within a predetermined pH range, and a water temperature within a predetermined water temperature range;
oxygen injection structure for injecting oxygen into the water in the receptacle interior to oxygenate the water;
a plant cutting support for supporting the plant cutting while maintaining the severed end thereof in the oxygenated water; and
water moving structure for causing continuous movement of the oxygenated water while maintaining the oxygenated water in engagement with the severed end of the supported plant cutting and within said predetermined pH and water temperature ranges.

13. The apparatus according to claim 12 wherein said receptacle has a receptacle bottom wall and incorporates downwardly extending projections, said downwardly extending projections maintaining more than a majority of said receptacle bottom wall out of engagement with a support surface supporting said receptacle to provide thermal insulation between said receptacle and said support surface.

14. The apparatus according to claim 12 wherein said water moving structure comprises a water pump employed to create a jet of water within the interior of the receptacle exerting water shear forces causing whirlpool motion of said water in said receptacle interior.

15. The apparatus according to claim 12 wherein said plant support includes a cover defining an opening disposed over said receptacle and a holder releasably connected to said cover at said opening.

16. The apparatus according to claim 14 wherein said oxygen injection structure includes an air inlet operatively associated with said water pump utilized to form air bubbles in said jet of water.

17. The apparatus according to claim 16 wherein the oxygen injection structure further includes a regulator for controlling air flow through the air inlet.
